(19) United States
(12) Patent Application Publication
(10) Pub. No.: US 2024/0090774 A1
(43) Pub. Date: Mar. 21, 2024

(54) SYSTEM FOR PREDICTING VASCULAR PLAQUE RUPTURE OR DETACHMENT THAT COULD LEAD TO A STROKE AND/OR FOR PREDICTING VASCULAR THROMBOSIS

(71) Applicants: OCTOGONE MEDICAL, Paris (FR); UNIVERSITE PARIS CITE, Paris (FR); INSTITUT NATIONAL DE LA SANTÉ ET DE LA RECHERCHE MÉDICALE, Paris (FR); ASSISTANCE PUBLIQUE HOPITAUX DE PARIS, Paris (FR)

(72) Inventors: Emmanuel MESSAS, Paris (FR); Frédéric SALDMANN, Paris (FR)

(21) Appl. No.: 18/262,953

(22) PCT Filed: Jan. 25, 2022
(86) PCT No.: PCT/FR2022/050130

§ 371 Date: Jul. 26, 2023

(30) Foreign Application Priority Data
Jan. 27, 2021 (FR) FR21 00754
Aug. 11, 2021 (FR) FR21 08631

(51) Int. Cl. A61B 5/02; A61B 5/00; A61B 5/053

(52) U.S. Cl. CPC A61B 5/02007; A61B 5/0015; A61B 5/053; A61B 5/6833; A61B 5/6867; A61B 5/7267; A61B 5/7275; A61B 2560/045; A61B 2560/0462; A61B 2562/0204; A61B 2562/0219; A61B 2562/0271

(57) ABSTRACT

System (1) for predicting an at least partial rupture or the detachment of a vascular plaque which could lead to a stroke or for analysing the biomechanical properties of the wall of an internal jugular vein in order to predict a risk of thrombosis, comprising: a monitoring device (2) comprising a temperature sensor (5) configured to measure the temperature of the vascular plaque or the wall of the jugular vein, a vibration sensor (4) configured to measure mechanical waves passing through the vascular plaque and the underlying walls, a motion sensor (6) configured to measure the movements of the plaque or the wall of the jugular vein (6), an electrical sensor configured to measure a parameter related to the electrical impedance of the plaque, an acoustic wave sensor and a calculation unit (10) configured to analyse the data from the monitoring device (2) by artificial intelligence.

### TECHNICAL FIELD

The present disclosure relates to a system for predicting rupturing or detachment of vascular plaque on the carotid or on supra-aortic trunks that can lead to a stroke.

More specifically, the present disclosure relates to a system and a method for ensuring continuous monitoring of the evolution of the stability of the vascular plaques present in the carotid or supra-aortic trunks of a patient and for predicting the occurrence of a stroke by rupturing vascular plaque. The system is also adapted to ensure continuous monitoring of the biomechanical properties of the internal jugular vein that crosses the carotid bifurcation in order to predict the occurrence of vascular thrombosis.

### PRIOR ART

A stroke is linked to a sudden interruption of the circulation of blood to all or part of the brain.

The brain is irrigated by the internal carotid and by all the supra-aortic trunks by supplying the oxygen necessary for its operation. A process known as atheroma causes thickening and hardening of the arteries by the accumulation, on the internal wall of the arteries, of fats, of complex carbohydrates, of blood and of calcareous deposits, and causes the progressive formation of one or more atheroma plaques. Other causes of vascular plaque formation also exist. It is a process whose evolution is accelerated by identified risk factors, namely arterial hypertension, diabetes, obesity, smoking. The presence of atheroma plaques thus causes a reduction in the diameter of the artery, which is called “stenosis”, a disease that is more generally called atherosclerosis. This plaque also can have a post-radial or post-inflammatory origin or can be due to a collagen disease. In all these cases, the risk of rupturing exists. The presence of the vascular plaque (due to atheroma or other origins) can be detected by various techniques, namely, vascular Doppler ultrasound, optical coherence tomography, computer-assisted tomography, or Magnetic Resonance Imaging.

A dramatic consequence related to rupturing of the plaque on the carotid or the supra-aortic trunks is expressed by the migration of certain components of the plaque in the circulation of blood to the brain, thus obstructing one of the cerebral arteries, causing an ischemic stroke. One of the other possible evolutionary risks of atheroma plaque, which is more rare, is rupturing of the plaque that results in the occlusion of the internal carotid, causing a hemodynamic stroke due to a low flow rate in the case of non-compensation of the Willis polygon. In each of the cases, the dramatic consequences are correlated with the detachment or rupturing of some or all the plaque. Rupturing some of the plaque or all the plaque is most often followed by a more or less calcified fibrinogen embolism that is able to obstruct the cerebral artery.

Current assessment, which is solely based on the degree of stenosis, is far from being sufficient and does not allow plaque rupturing and the actual risk of a stroke to be predicted. It is thus essential to be able to assess the risk of rupturing the atheroma plaque or other origins in order to precisely define the individual risk of a stroke occurring.

Currently, assessing the risk of rupturing vascular plaques is mainly based on the morphology and on the tissue composition of the plaque. Indeed, the morphological parameters, such as the thickness of the fibrous cap, the size of the necrotic body and the stenosis (the bulk of the arterial light), are considered to be factors influencing the occurrence of rupturing of the plaque. Plaques that have broken are characterized by a large fat core and a thin fibrous cap and intra-plaque hemorrhaging. However, these indicators do not allow reliable prediction of the risk of rupturing the vascular plaque.

An aim of the present disclosure is to propose a system that allows continuous monitoring of the evolution of the state of the integrity of the vascular plaque, in order to be able to predict the occurrence of a stroke caused by rupturing or detachment of plaque.

Another aim of the present disclosure is to propose a system and a method for predicting the occurrence of a stroke that is not very restrictive for the patient and can be easily used by the practitioner.

Another aim of the present disclosure is to propose a system and a method that allows continuous monitoring of the evolution of the biomechanical properties of an internal jugular vein in order to predict the risk of vascular thrombosis.

### SUMMARY

The present disclosure improves the situation.

A system is proposed for predicting an at least partial rupture or the detachment of vascular plaque that could lead to a stroke, with said vascular plaque being present on an arterial wall selected from among a carotid wall and a supra-aortic trunk wall, said system comprising:
a monitoring device able to be placed in the vicinity of the vascular plaque, said device comprising at least one temperature sensor configured to measure the temperature of the vascular plaque, at least one vibration sensor configured to measure mechanical waves propagated in said arterial wall, at least one motion sensor for the vascular plaque configured to measure movements of the vascular plaque, a memory able to store signals transmitted by said sensors, a communication interface, a power source configured to power said sensors and the communication interface;
a computation unit adapted to communicate with the monitoring device and configured to analyze measurements of the vibration sensor, of the temperature sensor and of the vascular plaque motion sensor originating from the monitoring device by artificial intelligence trained to detect whether there is a risk of at least partial rupturing or detachment of the vascular plaque.

According to one embodiment, the monitoring device further comprises at least one electrical sensor configured to measure a parameter related to the electrical impedance of the vascular plaque.

According to another embodiment, the monitoring device further comprises at least one acoustic wave sensor for measuring the acoustic waves originating from the arterial wall and/or from the vascular plaque.

By virtue of the monitoring device being directly attached to the patient, it is possible to monitor the evolution of the integrity of the vascular plaques in real time.

The system allows the risk of the occurrence of a stroke to be predicted without requiring complex and often invasive examinations of the patient.

The features disclosed in the following paragraphs optionally can be implemented independently of one another or in combination with one another:

The monitoring device is in the form of a patch able to be adhered to an external surface of the skin of a patient.

The monitoring device is in the form of a subcutaneous implant able to be inserted under the skin of a patient.

The temperature sensor is a sensor for detecting thermal waves emitted by the vascular plaque.

The vibration sensor comprises an accelerometer.

The vibration sensor comprises a 3-axis accelerometer and a 3-axis gyroscope.

The vascular plaque motion sensor is an ultrasound probe.

The power source is an induction-rechargeable battery.

The communication interface is selected from among a short-range radio interface or a near-field communication interface.

According to another embodiment, the system further comprises at least one mobile communication appliance adapted to remotely communicate with the monitoring device via said communication interface, which is a short-range interface, and to transmit signals to the computation unit via a long-range communication interface forming part of the computation unit.

According to one embodiment, the monitoring device comprises at least one temperature sensor and/or at least one motion sensor and/or at least one acoustic wave sensor, with the measurement frequency of said sensors being set so as to transmit and/or receive a signal originating from an internal jugular vein.

According to another aspect, a method is proposed for predicting an at least partial rupture or the detachment of a vascular plaque that can lead to a stroke, with said vascular plaque being present on an arterial wall selected from among a carotid wall and a supra-aortic trunk wall of a patient using a system as described above, the method comprising;
continuously acquiring, for a determined period of time, a plurality of signals representing the evolution of the risk of rupturing or detachment of the vascular plaque by means of the monitoring device placed in the vicinity of the vascular plaque;
analyzing the plurality of signals in a computation unit by artificial intelligence trained to detect the risk of an at least partial rupture or the detachment of the vascular plaque.

According to one embodiment, the artificial intelligence is a neural network and the method further comprises a prior learning step comprising;
acquiring a plurality of signals, called reference signals, originating from monitoring devices worn by a population with known risks of rupturing or detachment of the vascular plaque;
training the neural network with said reference signals until it converges.

### BRIEF DESCRIPTION OF THE DRAWINGS

Further features, details and advantages will become apparent upon reading the following detailed description, and with reference to the appended drawings, in which:

FIG. 1 is an illustration of a system for predicting rupturing or detachment of vascular plaque that can lead to a stroke according to one embodiment.

FIG. 2 is an illustration of a prediction system according to another embodiment.

FIG. 3 shows the monitoring device of the prediction system positioned on the neck of a patient, in the vicinity of the internal carotid after the bifurcation of the common carotid, facing the vascular plaque.

FIG. 4 shows an embodiment of the monitoring device attached to an external surface of the skin, in the vicinity of the carotid.

FIG. 5 shows another embodiment of the monitoring device implanted under the skin, in the vicinity of the carotid.

FIG. 6 is a flowchart representing the prediction method implemented by the prediction system according to one embodiment.

FIG. 7 is a flowchart representing the learning step for training the neural network used by the computation unit.

FIG. 8 is a schematic illustration of the monitoring device further comprising an electrical sensor configured to measure a parameter related to the electrical impedance of the vascular plaque according to one embodiment.

FIG. 9 is a schematic illustration of the monitoring device further comprising an acoustic wave sensor configured to measure acoustic waves originating from the arterial wall and/or the vascular plaque.

### DESCRIPTION OF THE EMBODIMENTS

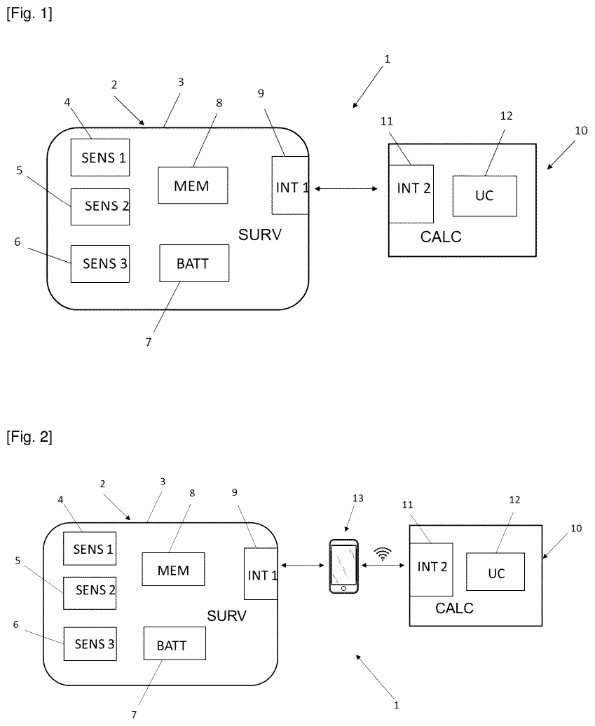

FIG. 1 illustrates an embodiment of a system 1 for predicting a risk of rupturing or detachment of a vascular plaque that can lead to a stroke by rupturing plaque. The system 1 comprises:
a monitoring device 2 (SURV) configured to be disposed in the vicinity of the carotid or the supra-aortic trunks of a patient in order to measure the signals representing the mechanical stability of one or more vascular plaques (of atheroma or other origins) formed on the internal wall of the carotid; and
a computation unit 10 (CALC) adapted to communicate with the monitoring device 2 and configured to analyze the signals originating from the monitoring device 2 in order to detect whether there is a risk of rupturing at least a portion of the vascular plaque. Typically, the computation unit 10 can be operated by a practitioner or a group of practitioners, and can gather and process the data from a large number of monitoring devices 2.

The monitoring device 2 can comprise:
one or two vibration sensors 4 (SENS 1), notably one or more accelerometers, configured to measure mechanical waves that can be generated by the arterial pulse wave passing through the vascular plaque and the underlying walls;
a temperature sensor 5 (SENS 2) configured to measure the temperature of the vascular plaque; and
a plaque motion sensor 6 (SENS 3), notably an ultrasound transducer, such as a piezoelectric crystal, configured to measure the movements of the vascular plaque.

The monitoring device 2 also comprises a memory 8 (MEM) able to store the signals transmitted by the sensors 4, 5, 6 and a communication interface 9 (INT 1). The communication interface 9 can be, for example:
a short-range radio interface, notably of the “Bluetooth”, “Wi-Fi” or other type;
or even a near-field communication interface, notably of the NFC or RFID type.

The signals stored in the memory 8 can be transmitted to the computation unit 10 via the communication interface 9.

The computation unit 10 is configured to analyze the signals, notably by trained artificial intelligence, and to detect the occurrence of a stroke by vascular plaque rupturing. The computation unit 10 comprises, for example, a central unit 12 (UC), which stores the trained neural network. The computation unit 10 also comprises a communication interface 11 (INT 2) for receiving the signals originating from the monitoring device 2. The communication interface 11 and the central unit 12 optionally can be remote from each other. The central unit 12 optionally can be a server.

The function of the sensors 4, 5 and 6 is described hereafter.

Rupturing or detachment of the vascular plaque attached to the internal wall of the artery can be caused by a mechanical wave generated, for example, by the arterial pulse wave. The mechanical wave comprises a shear component and a compression component, and can be equated with a seismic wave. By measuring the mechanical waves, the vibration sensor 4 is able to measure a signal representing the biomechanical properties of the vascular plaque and of the underlying walls. The vibration sensor 4 can comprise at least one accelerometer. For example, the vibration sensor can comprise a 3-axis accelerometer and a 3-axis gyroscope. The data representing the signal measured by the vibration sensor 4 are stored in the memory 8.

The temperature sensor 5 is able to measure a local temperature variation representing the stability of the plaque. Indeed, inflammation is one of the main features of an unstable plaque. The intensity of the inflammatory process results in an increase in temperature in the plaque. According to one embodiment, the temperature sensor 5 can be configured to measure a thermal wave emitted by the plaque in the microwave frequency domain. Such a sensor can comprise, for example, an antenna for detecting said thermal waves emitted by the vascular plaque. The data representing the signal measured by the temperature sensor 5 are stored in the memory 8.

The plaque motion sensor 6 is a piezoelectric sensor configured to send ultrasound in order to detect the movements of the plaque in real time and to determine whether they are regular or irregular and uncontrolled, which results in an arrhythmia or a phenomenon disrupting the arterial pulse wave. The data representing the signal measured by the motion sensor 6 are stored in the memory 8.

With reference to FIG. 2, a prediction system according to another embodiment is described. It further comprises a mobile communication appliance 13 communicating with the monitoring device 2 via a short-range communication interface. The mobile communication appliance 13 comprises an application, via which the patient, wearing the monitoring device, can retrieve the signals stored in the memory 8. The monitoring device transmits the signals to the mobile communication appliance 13 via its communication interface 9, which is a short-range interface.

The mobile communication appliance 13 can also communicate with the computation unit 10. The mobile communication appliance can transmit, for example, the retrieved signals via a 2G, 3G, 4G or 5G type communication interface. The computation unit 10 can retrieve the signals via its communication interface 11, which is a long-range interface.

The mobile communication appliance 13 can be a smart phone or a smart watch, for example.

The monitoring device 2 is an autonomous device. The monitoring device 2 comprises a power source 7 (BATT), which, for example, can be recharged by an external device, notably by induction. The battery and the electronic components of the data acquisition device can be selected so that the autonomy of the device is at least equal to one month.

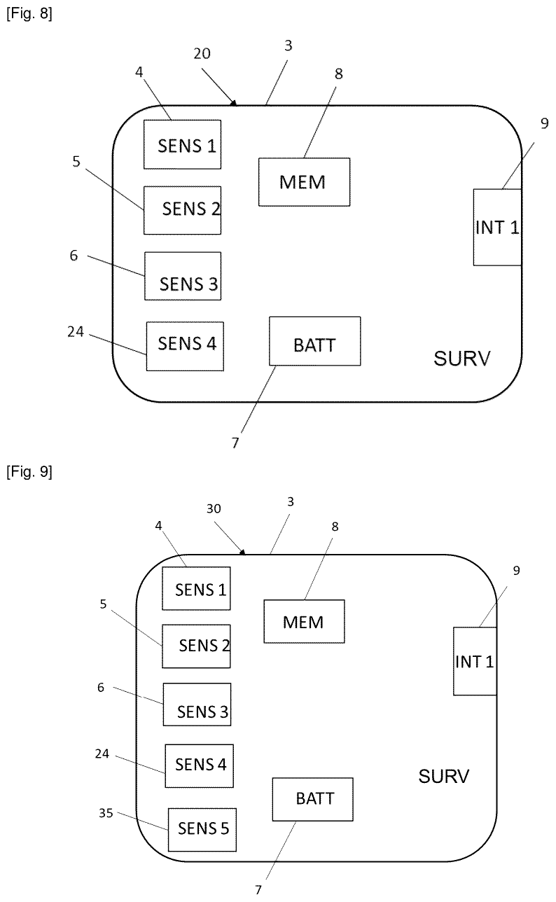

FIG. 8 illustrates a schematic view of a monitoring device 20 according to another embodiment, in which it comprises, in addition to the other three sensors 4, 5, 6 described above, a fourth sensor, which is an electrical sensor 24 (SENS 4).

The electrical sensor 24 is configured to measure the electrical impedance of the vascular plaque that expresses a level of stiffness of the plaque that has been identified as influencing the stability of the vascular plaque. The vascular plaque and the surrounding environment act as a dielectric medium. By measuring the impedance, it is possible to extract the dielectric parameters, namely the conductivity and the permittivity of this medium, representing the level of stiffness of the plaque. According to one embodiment, the electrical impedance sensor is configured to apply a current and to measure a potential difference. Knowing the voltage and the current, the value of the impedance of the vascular plaque is determined based on Ohm's law. Continuous measurement of the impedance allows the evolution of the state of stiffness of the plaques to be monitored. The data representing the signal measured by the impedance sensor 24 are stored in the memory 8.

FIG. 9 illustrates a schematic view of a monitoring device 30 according to another embodiment, in which it comprises, in addition to the four sensors 4, 5, 6, 24 of FIG. 8, a fifth passive acoustic wave sensor 35 configured to measure the acoustic waves originating from the arterial wall and from the plaque.

It should be noted that the prediction system can also predict a risk of rupturing or detachment of a vascular plaque on the basis of one or more signals measured by the five sensors 4, 5, 6, 24, 35 of the monitoring device. The monitoring device can therefore comprise any one or more, or any combination from among the five sensors 4, 5, 6, 24, 35 described above for measuring a signal representing the mechanical stability of the vascular plaque.

According to one embodiment, the monitoring device comprises only one of the four sensors from among the five sensors 4, 5, 6, 24, 35 described above.

According to another embodiment, the monitoring device can comprise a combination of two sensors from among the five sensors 4, 5, 6, 24, 35 described above.

According to yet another embodiment, the monitoring device can comprise a combination of three sensors from among the five sensors 4, 5, 6, 24, 35 described above.

According to yet another embodiment, the monitoring device can comprise a combination of four sensors from among the five sensors 4, 5, 6, 24, 35 described above.

Advantageously, the measurement frequency of the sensors of the monitoring device can be set to transmit and/or receive signals originating from a region of interest located at a certain depth under the skin.

The combination of the sensors varies as a function of the clinical requirements.

Within the context of using the monitoring device to monitor the evolution of the biomechanical properties of a vascular plaque and/or of an underlying wall of the carotid over time, the monitoring device can comprise, for example, a vibration sensor 4 and/or a temperature sensor 5 and/or a motion sensor 6 and/or an electrical sensor 24 and/or an acoustic wave sensor 35 configured to transmit and/or receive signals originating from a vascular plaque and/or from an underlying wall of the carotid, which are located at a variable depth from the surface of the skin, generally ranging between 1-3 cm under the skin. The frequency of the measurements of the vibration sensor 4, of the temperature sensor 5, of the motion sensor 6, of the electrical sensor 24 and of the acoustic wave sensor 35 are set to transmit and/or receive the signal originating from the vascular plaque and/or the underlying wall of the carotid.

Within the context of using the monitoring device to measure the changes of the biomechanical properties of the internal jugular vein, the monitoring device can comprise, for example, a temperature sensor 5 and/or a motion sensor 6 and/or an acoustic wave sensor 35 configured to transmit and/or receive signals originating from an internal jugular vein that is located at a variable depth from the surface of the skin depending on the anatomy of the patient, generally ranging between 1 and 3 cm under the skin. The frequency of the measurements of the temperature sensor 5, of the motion sensor 6 and of the acoustic wave sensor 35 are different and are set to transmit and/or receive the signal originating from the jugular vein.

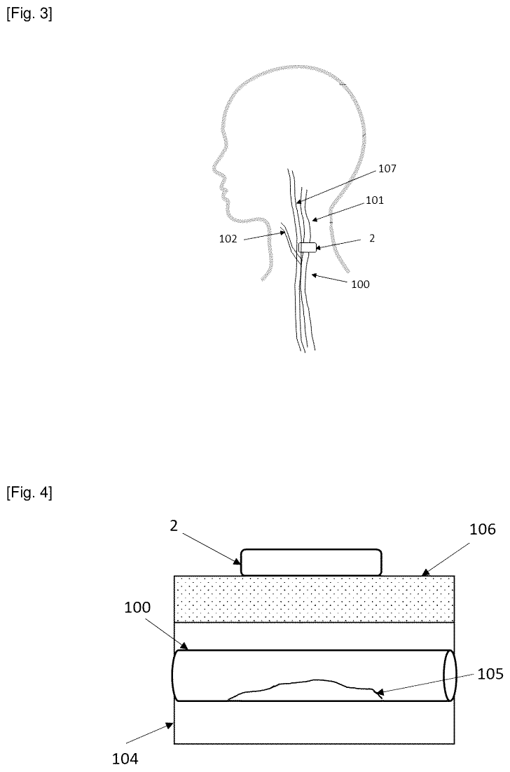

FIG. 3 illustrates a schematic view of a common carotid 100 that splits into two at the neck of a patient, namely: the internal carotid artery 101, which will irrigate the brain, and the external carotid artery 102, which will irrigate the neck and the face. The monitoring device 2 as described above can be placed facing the internal carotid immediately after the bifurcation of the common carotid, so as to continuously measure the various signals transmitted by one or more plaques present on the internal wall of the internal carotid.

FIG. 3 also schematically illustrates an internal jugular vein 107, which is a deep vein of the neck, in contact with the internal carotid artery. There is an internal jugular vein on each side of the neck. The internal jugular vein runs vertically downward, lateral to the internal carotid artery, then to the common carotid artery.

In general, the positioning of the device can be set as a function of the clinical requirements. According to another embodiment, it is also possible to position a second monitoring device or even several devices in order to monitor not only the internal carotid, but also its branches or any other artery of the supra-aortic trunks. By way of an example, it is possible to position a second monitoring device immediately before the bifurcation of the common carotid 100.

With reference to FIG. 4, the monitoring device 2 is in the form of a patch 3 having an adhesive surface that allows the device to be adhered to an area of the outer surface of the skin 106 of the neck, in the vicinity of the carotid 100.

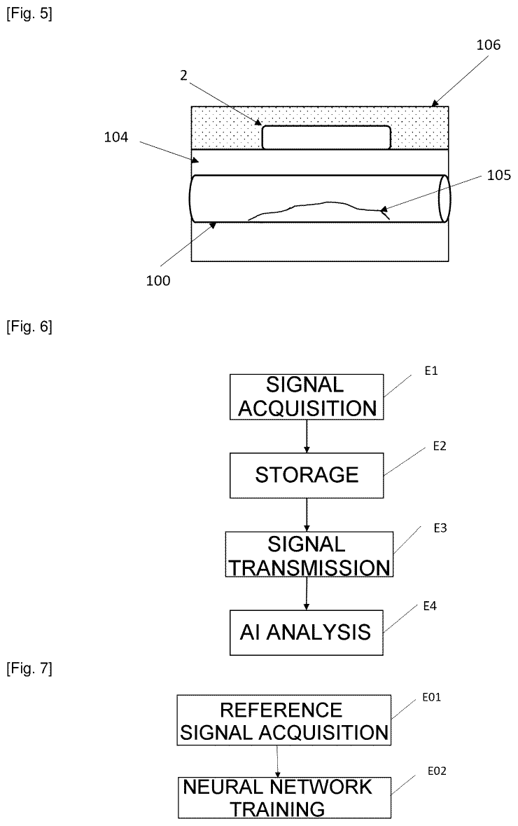

With reference to FIG. 5, the monitoring device 2 also can be a subcutaneous implantable device. The monitoring device 2 comprises, for example, a casing made of a biocompatible material. The monitoring device is placed in an implantable probe. It can be placed under the skin 106, in the vicinity of the carotid 100. It is fitted by the practitioner, who creates a detachment under the skin. The device is fitted in contact with the muscle 104 under the skin.

One aspect of the present disclosure is to allow the system 1 to be used to continuously monitor the changes of the biomechanical properties of an internal plaque and/or of an underlying wall of the carotid. Within the context of this use, the monitoring device can comprise a vibration sensor 4 and/or a temperature sensor.

The monitoring method can be implemented in a non-invasive mariner by fitting the monitoring device on the skin of a patient, in the vicinity of the carotid.

The monitoring method also can be implemented by fitting the monitoring device under the skin of a patient, in the vicinity of the carotid.

With reference to FIG. 6, a method implementing a system for predicting a risk of rupturing or detachment of plaque that can lead to a stroke is described hereafter.

In step E1, the vibration sensor 4 and/or the temperature sensor 5 and/or the motion sensor 6 and/or the electrical sensor 24 and/or the acoustic wave sensor 35 are set so as to operate in a range of frequencies adapted for transmitting and/or receiving signals originating from a vascular plaque and/or from an underlying wall of the carotid.

In step E1, the vibration sensor 4 and/or the temperature sensor 5 and/or the motion sensor 6 and/or the electrical sensor 24 and/or the acoustic wave sensor 35 of the monitoring device measure the signals originating from the plaque for a predetermined period of time. The duration varies from a few hours to a few weeks. The sensors are, for example, previously programmed to operate for a determined duration. This duration is determined by the practitioner as a function of the clinical requirements.

According to one embodiment, the three sensors 4, 5 and 6 of the monitoring device measure the signals originating from the plaque and/or the wall of the carotid for a determined duration.

According to another embodiment, the four sensors 4, 5, 6, 24 of the monitoring device measure the signals originating from the plaque and/or the wall of the carotid for a determined duration.

According to yet another embodiment, the five sensors 4, 5, 6, 24, 35 of the monitoring device measure the signals originating from the plaque and/or the wall of the carotid for a determined duration.

In step E2, the signals are stored in the memory 8.

In step E3, the signals stored in the memory 8 of the monitoring device are sent to the computation unit 10 via the communication interface 9.

In step E4, the computation unit 10 analyzes the signals by means of artificial intelligence trained to detect a risk of rupturing or detachment of plaque that can lead to a stroke. The artificial intelligence comprises a neural network trained to determine, on the basis of the gathered signals, whether the plaque(s) present in the carotid exhibit a risk of rupturing.

Steps E3 and E4 are carried out in the practice of the practitioner, for example. It is thus possible for the practitioner to acquire a reliable prediction of the risk of a stroke occurring by rupturing the plaque in the patient, even if the patient exhibits no symptoms of the pathology.

As an alternative embodiment, in step E3, the patient can also use a mobile communication appliance 13 in order to periodically communicate with the monitoring device via a short-range communication interface. The appliance is a smartphone, for example. It comprises, for example, an application by which the patient can poll the monitoring device in order to receive the signals stored in the memory 8 of the monitoring device. The communication appliance 13 then transmits the signals, via a 4G or 5G network, to the computation unit 10. Thus, a diagnosis of the evolution of the state of stability of the plaques can be remotely and periodically established by the practitioner, for example, once a week.

According to one embodiment, and with reference to FIG. 7, when the artificial intelligence is a neural network, the method can further comprise a preliminary learning step for training the artificial intelligence so as to determine, on the basis of the gathered signals, whether the plaque(s) present in the carotid exhibit a risk of rupturing or detachment.

More specifically, the learning step can comprise the following sub-steps.

In a sub-step E01, a plurality of monitoring devices is used to gather signals from a population with a known risk of rupturing the vascular plaque. The signals, called reference signals, are stored on a server.

In a sub-step E02, the neural network is trained with the reference signals until it converges. The trained neural network is then stored in the computation unit 10, notably in the central unit 12.

By virtue of the continuous acquisition of a set of signals representing the evolution of the stability of the vascular plaque and of the use of artificial intelligence to analyze the acquired signals, the system of the present disclosure enables prediction of the risk of rupturing plaque that can lead to the occurrence of a stroke. The present system can be used by the practitioner, in addition to morphological and histological studies, to monitor and locally assess the evolution of the risk of rupturing plaques and thus prevent serious clinical consequences such as a stroke. The system of the present disclosure thus allows individuals who require interventions to be identified quickly, even in the absence of pathological symptoms.

Another aspect of the present disclosure is to allow the system to be used to continuously monitor changes in the biomechanical properties of an internal jugular vein that is located deeper under the skin relative to the carotid. Within the context of this use, the monitoring device can comprise a temperature sensor 5, a motion sensor 6 and an acoustic wave sensor 35.

The monitoring method can be implemented in a non-invasive manner by fitting the monitoring device on the skin of a patient, in the vicinity of the internal jugular vein.

The monitoring method also can be implemented by fitting the monitoring device under the skin of a patient, in the vicinity of the internal jugular vein.

With reference to FIG. 6, in step E1, the temperature sensor 5, the motion sensor 6 and the acoustic wave sensor 35 of the monitoring device 2 are set so as to operate in a suitable frequency range that allows signals originating from the internal jugular vein to be transmitted and/or received for a determined duration.

The duration of the measurement varies from a few hours to a few weeks. The sensors are, for example, previously programmed to operate for a determined duration. This duration is determined by the practitioner depending on the clinical requirements. The gathered signals represent any changes of the biomechanical properties of the internal jugular vein over time.

In step E2, the signals are stored in the memory 8.

In step E3, the signals stored in the memory 8 of the monitoring device are sent to the computation unit 10 via the communication interface 9.

In step E4, the computation unit 10 analyzes the signals by means of artificial intelligence trained to detect a risk of vascular thrombosis. The artificial intelligence comprises a neural network trained to determine, on the basis of the gathered signals, whether the changes of the biomechanical properties of the internal jugular vein exhibit a risk of vascular thrombosis.

Steps E3 and E4 are carried out in the practice of the practitioner, for example. It is thus possible for the practitioner to acquire a reliable prediction of the risk of vascular thrombosis occurring in the patient, even if the patient exhibits no symptoms of the pathology.

As an alternative embodiment, in step E3, the patient can also use a mobile communication appliance 13 in order to periodically communicate with the monitoring device via a short-range communication interface. The appliance is a smartphone, for example. It comprises, for example, an application by which the patient can poll the monitoring device in order to receive the signals stored in the memory 8 of the monitoring device. The communication appliance 13 then transmits the signals, via a 4G or 5G network, to the computation unit 10. Thus, the evolution of the biomechanical properties of the internal jugular vein can be monitored remotely and periodically by the practitioner, for example, once a week.

According to one embodiment, and with reference to FIG. 7, when the artificial intelligence is a neural network, the method can further comprise a preliminary learning step for training the artificial intelligence so as to determine, on the basis of the signals gathered over time, how to characterize a risk of the occurrence of vascular thrombosis.

More specifically, the learning step can comprise the following sub-steps.

In a sub-step E01, a plurality of monitoring devices is used to gather signals from a population with a known risk of vascular thrombosis. The signals, called reference signals, are stored on a server.

In a sub-step E02, the neural network is trained with the reference signals until it converges. The trained neural network is then stored in the computation unit 10, notably in the central unit 12.

By virtue of the continuous acquisition of a set of signals representing the evolution of the changes of the biomechanical properties of the internal jugular vein and of the use of artificial intelligence to analyze the acquired signals, the system of the present disclosure also enables prediction of the risk of a blood clot forming in the internal jugular vein of the neck, which can lead to the occurrence of vascular thrombosis. The method described hereafter allows the practitioner to be assisted, in addition to known examinations, for example, a Doppler-echo, which allows the clot to be viewed, in assessing the risk of jugular thrombosis in order to be able to quickly take care of the patient.

Furthermore, the use of the system is not only limited to predicting a stroke. It can be fitted in a patient after a stroke has occurred in order to predict the risk of recurrence following treatment of the vascular plaques.

Although the system of the present invention has particular advantages for predicting the occurrence of a stroke, the system can be generally applied for the early detection of patients at risk of developing cardiovascular diseases. The system also can be applied during a clinical trial for assessing the effectiveness of a new treatment of vascular plaques.

1. A system for predicting an at least partial rupture or a detachment of vascular plaque that could lead to a stroke, with said vascular plaque being present on an arterial wall selected from among a carotid wall and a supra-aortic trunk wall, said system comprising:
a monitoring device able to be placed in a vicinity of the vascular plaque, said monitoring device comprising
at least one temperature sensor configured to measure temperature of the vascular plaque,
at least one vibration sensor configured to measure mechanical waves propagated in said arterial wall,
at least one motion sensor for the vascular plaque configured to measure movements of the vascular plaque,
a memory able to store signals transmitted by one or more of said at least one temperature sensor, said at least one vibration sensor, and said at least one motion sensor,
a communication interface, and
a power source configured to power the communication interface and one or more of said at least one temperature sensor, said at least one vibration sensor, and said at least one motion sensor; and
a computation unit adapted to communicate with the monitoring device and configured to analyze measurements of the at least one vibration sensor, of the at least one temperature sensor, and of the at least one motion sensor originating from the monitoring device by artificial intelligence trained to detect whether there is a risk of at least partial rupturing or detachment of the vascular plaque.

2. The system as claimed in claim 1, wherein the monitoring device further comprises at least one electrical sensor configured to measure a parameter related to the electrical impedance of the vascular plaque.

3. The system as claimed in claim 1, wherein the monitoring device further comprises at least one acoustic wave sensor for measuring acoustic waves originating from the arterial wall and/or from the vascular plaque.

4. The system as claimed in claim 1, wherein the monitoring device is a patch able to be adhered to an external surface of skin of a patient.

5. The system as claimed in claim 1, wherein the monitoring device is a subcutaneous implant able to be inserted under skin of a patient.

6. The system as claimed in claim 1, wherein the at least one temperature sensor is configured for detecting thermal waves emitted by the vascular plaque.

7. The system as claimed in claim 1, wherein the at least one vibration sensor comprises an accelerometer.

8. The system as claimed in claim 7, wherein the vibration sensor comprises a 3-axis accelerometer and a 3-axis gyroscope.

9. The system as claimed in claim 1, wherein the at least one motion sensor is an ultrasound probe.

10. The system as claimed in claim 1, wherein the power source is an induction-rechargeable battery.

11. The system as claimed in claim 1, wherein the communication interface is selected from the group consisting of a short-range radio interface and a near-field communication interface.

12. The system as claimed in claim 1, further comprising at least one mobile communication appliance adapted to remotely communicate with the monitoring device via said communication interface, wherein said communication interface is a short-range interface, and wherein said at least one mobile communication appliance is configured to transmit signals to the computation unit via a long-range communication interface which forms at least forming part of the computation unit.

13. The system as claimed in claim 3, wherein a measurement frequency of one or more of said at least one temperature sensor, said at least one vibration sensor, and said at least one motion sensor is set so as to transmit and/or receive a signal originating from an internal jugular vein.
